(19) United States
(12) Patent Application Publication
(10) Pub. No.: US 2013/0224294 A1
(43) Pub. Date: Aug. 29, 2013

(54) DRY PROCESSING OF ATAZANAVIR

(71) Applicants: Dominique Meergans, Munich (DE); Ralph Stefan, Ebenweiler (DE)

(72) Inventors: Dominique Meergans, Munich (DE); Ralph Stefan, Ebenweiler (DE)

(73) Assignee: RATIOPHARM GMBH, Ulm (DE)

(21) Appl. No.: 13/876,236

(22) PCT Filed: Sep. 27, 2011
(86) PCT No.: PCT/EP2011/004835

§ 371 Date: May 9, 2013

(30) Foreign Application Priority Data
Sep. 28, 2010 (DE) 10 2010 046 779.0

(51) Int. Cl. A61K 9/00; A61J 3/10

(52) U.S. Cl. CPC A61K 9/0002; A61J 3/10
USPC 424/464; 514/21.91; 424/474; 264/109; 427/2.14

(57) ABSTRACT

The invention relates to dry processes for producing oral dosage forms, more specifically tablets, comprising atazanavir and adhesion enhancers. The invention further relates to compacted intermediates comprising atazanavir and adhesion enhancers.

The invention relates to dry processes for producing oral dosage forms, more specifically tablets, comprising the active compound atazanavir and adhesion enhancers. The invention further relates to compacted intermediates comprising atazanavir and adhesion enhancers.

The IUPAC name of atazanavir [INN] is dimethyl N-[(1S)-1-{[(2S,(3S)-3-hydroxy-4-[(2S)-2-[(methoxycarbonyl)amino]-3,3-dimethyl-N-{[4-(pyrididin-2-yl)phenyl]methyl}-butanehydrazido]-1-phenylbutan-2-yl]carbonoyl}-2,2-dimethylpropyl]carbamate. Atazanavir, formerly also referred to as BMS-232632, is classified as a BCS II drug (high permeability/low solubility). The chemical structure of atazanavir is represented by the following formula (1):

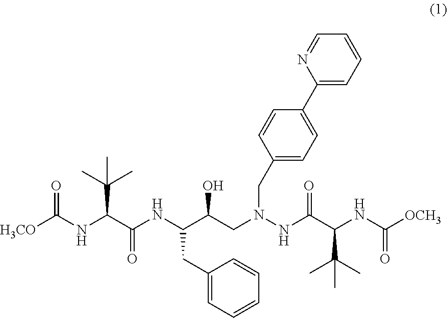

Synthetic pathways for atazanavir and its use as HIV protease inhibitor have been described in WO 97/40029. HIV protease inhibitors specifically inhibit HIV protease. The latter is a viral enzyme which cleaves a large precursor protein into various proteins important to the virus during the late phase of the viral propagation cycle. Protease inhibitors prevent this cleavage and, as a result, cause the formation of defective viral particles.

The free base of atazanavir does not have sufficient bioavailability. Therefore, quite a number of different acid addition salts such as, for example, the hydrochloride, methanesulphonate (mesylate), sulphate and bisulphate salts have been tested for the purpose of developing an orally administrable drug form. Owing to its good solubility in comparison with the other salts, atazanavir bisulphate is used for producing the currently available oral drug forms. The chemical structure of atazanavir bisulphate is represented by the following formula (2):

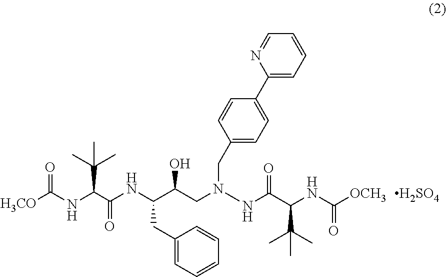

Atazanavir is commercially available under the tradenanme REYATAZ® from Bristol-Myers Squibb for the treatment of HIV. It is preferably in the form of capsules in dosage units of 100 mg, 150 mg, 200 mg and 300 mg of atazanavir which contain wet-granulated active compound.

Crystalline atazanavir, in particular in the form of the bisulphate salt, exhibits polymorphism. W099/36404 A1 discloses both anhydrous/desolvated type I crystals and hydrated, hygroscopic type II crystals of the bisulphate. WO2005/108349 A2 additionally makes mention of the forms A, E3 and C (referred to as “Pattern C”).

There are several possibilities of improving the bioavailability for drugs of the “BCS II” class. An obvious, and therefore very widespread, method comprises providing from the solution a uniform distribution of the active compound in the formulation.

WO99/36404 A1 and WO2005/108349 A2 disclose atazanavir in the form of capsules. Said capsules can be obtained by wet-granulating atazanavir bisulphate in combination with lactose, crospovidone and magnesium stearate. The production of tablets described in WO2009/002823 A2 is also performed with the aid of wet granulation.

Wet granulation of atazanavir bisulphate is necessary for achieving a satisfactory release behaviour (see EMEA 2005, Scientific Discussion REYATAZ®) since wet granulation results in crystalline atazanavir being converted predominantly (but not completely) to amorphous atazanavir. However, wet granulation is disadvantageous in that the amount of water used for granulation must be set very precisely because otherwise there is a risk of the salt dissociating in the presence of water, ultimately producing a relatively large amount of the virtually insoluble base (<1 μg/ml at 24±3° C.) (see differences in the solubility behaviour in Scientific Discussion). In addition, processing atazanavir bisulphate by means of wet granulation normally requires an active compound that has been produced by a special crystallization method and has a particularly narrow particle size distribution.

Moreover, there is evidence that the storage stability of the formulations disclosed in the prior art can be improved. This is because said formulations disclosed in the prior art exhibit undesired fluctuations in the release behaviour after storage (presumably due to conversion of different polymorphic forms and partial amorphization). This may result in a different release profile and therefore in an undesired irregular rise in the level of the active compound in the patient.

The capsules currently on the market have the disadvantage that absorption of atazanavir can be reduced if the pH in the stomach has increased, independently of the cause of said increase. Therefore, for example, taking atazanavir together with proton pump inhibitors is not recommended. (See “Summary of Product Characteristics” at the EMA).

It was therefore an object of the present invention to overcome the abovementioned disadvantages.

More specifically, it was intended to provide oral dosage forms of atazanavir which have an advantageous release profile compared to the oral dosage forms of the prior art. The release profile here should be advantageous especially after storage.

It was therefore an object of the invention to provide atazanavir in a form which enables its level in the patient to rise as steadily as possible. Both interindividual and intraindividual differences should substantially be avoided.

Another object of the invention was to provide a process for producing atazanavir-containing oral dosage forms, which also enables particulate atazanavir that does not have a narrow particle size distribution to be used.

Finally, it was an object of the invention to provide a process for producing atazanavir-containing tablets which exhibit advantageous coatability. Coating of the tablets should not produce any “flaking”.

Unexpectedly, the above objects were achieved by dry processing of atazanavir together with a special amount of an adhesion enhancer.

The invention therefore relates to a process for producing oral dosage forms, more specifically tablets, comprising atazanavir and adhesion enhancers, wherein said dosage forms are produced by means of dry compaction or by means of direct compression, and the atazanavir to adhesion enhancer weight ratio is from 1:10 to 10:1, preferably 5:1 to 1:7.

The invention furthermore relates to tablets which can be produced by the process according to the invention.

The invention further relates to an intermediate obtainable by dry compaction of atazanavir together with an adhesion enhancer.

Finally, the invention relates to single and multiple dose containers, preferably sachets and stick packs, comprising the intermediate according to the invention.

Oral dosage forms for the purpose of the present invention comprise capsules, tablets, pellets, granules or powders.

In the context of the present invention, the term “atazanavir” comprises dimethyl (3S,8S,9S,12S)-3,12-bis(1,1 -dimethylethyl)-8-hydroxy-4,11 -dioxo-9-(phenylmethyl)-6-[[4-(2-pyridinyl)phenyl]methyl]-2,5,6,10,13-pentaazatetradecanedioate according to formula (1) above, and its solvates and hydrates. Moreover, the term “atazanavir” also comprises all pharmaceutically compatible salts and their solvates and hydrates.

The salts may be acid addition salts. Examples of suitable salts are hydrochlorides (monohydrochloride, dihydrochloride), carbonates, hydrogencarbonates, acetates, lactates, butyrates, propionates, sulphates, methanesulphonates, citrates, tartrates, nitrates, sulphonates, oxalates and/or succinates. Preference is given to using atazanavir bisulphate. Atazanavir bisulphate is a salt of atazanavir base and H₂SO₄, with the molar ratio, as depicted in formula (2), being 1:1.

Preference is given to employing atazanavir in the A form described in WO 2005/108349 A2.

One embodiment of the present invention makes use of particulate atazanavir, with the average particle diameter, i.e. the particle size distribution D₅₀ value, being from 1 to 200 μm, preferably from 3 to 100 μm, more preferably from 5 to 70 μm, still more preferably 7 to 50 μm, particularly preferably 10 to 40 μm, in particular 12 to 30 μm.

“Particle diameter” or “particle size” of a particle to be determined means according to the invention the diameter of an equivalent particle which is assumed to be spherical and to have the same light scattering pattern as the particle to be determined. According to the invention, particle size is determined by means of laser diffractometry. More specifically, the particle size was determined using a Mastersizer 2000 from Malvern Instruments. Preference is given to carrying out a wet measurement using a dispersion in a dispersant, 1750 rpm and ultrasound for 30 s. Particles with a D₅₀ value of less than 5.0 μm are evaluated with the aid of the Mie method, and particles with a D₅₀ value of 5.0 μm or larger are evaluated with the aid of the Fraunhofer method.

“Particle size distribution” here means the statistical distribution of the partial volumes based on all available particle sizes of the sample measured. “Partial volume” means the volume-based percentage of all particles having a defined particle size.

According to the invention, the particle size distribution D₅₀ value describes the particle size at which 50% by volume of the particles have a smaller particle size than the particle size corresponding to the D₅₀ value. Likewise, 50% by volume of said particles then have a larger particle size than the D₅₀ value.

Accordingly, the D₉₀ value of the particle size distribution of the intermediate is defined as the particle size at which 90% by volume of the particles have a smaller particle size than the particle size corresponding to the D₉₀ value.

Similarly, the D₁₀ value of the particle size distribution of the intermediate is defined as the particle size at which 10% by volume of the particles have a smaller particle size than the particle size corresponding to the D₁₀ value.

Preferably, the atazanavir used in the process according to the invention and in the intermediate according to the invention has generally a D₉₀ value of from 3 to 350 μm, preferably from 5 to 150 μm, more preferably from 10 to 100 μm, particularly preferably from 15 to 80 μm.

Preferably, the atazanavir used in the process according to the invention and in the intermediate according to the invention has generally a D₅₀ value of 2-50 μm, preferably of 5-30 μm and particularly preferably of 7-20 μm.

Preferably, the atazanavir used in the process according to the invention and in the intermediate according to the invention has generally a D₁₀ value of from 0.1 to 100 μm, preferably from 0.5 to 50 μm, more preferably from 1.0 to 25 μm, particularly preferably from 2.0 to 15 μm.

In a further preferred embodiment, the ratio between the D₉₀ value and the D₅₀ value (=D₉₀/D₅₀) of atazanavir has a value of between 1.1 and 8.0, preferably between 1.2 and 4.0, particularly preferably between 1.3 and 2.5. In a further preferred embodiment, the ratio between the D₅₀ value and the D₁₀ value (=D₅₀/D₁₀) of atazanavir has a value of between 1.1 and 8.0, preferably between 1.2 and 4.0, particularly preferably between 1.3 and 2.5.

In a preferred embodiment, the atazanavir used, or alternatively its pharmaceutically compatible salt, more specifically atazanavir bisulphate, has a water content of from 0.01 to 10% by weight, more preferably from 0.05 to 8.0% by weight, particularly preferably from 0.1 to 3.0% by weight. In the context of the present application, the water content is determined preferably by the Karl Fischer method, using a coulometer at 130° C. Preference is given to using a Karl-Fischer Titrator Aqua 40.00 from ECH (Electrochemie Halle).

Usually, a sample of from 20 to 70 mg of atazanavir is analyzed.

The oral dosage form according to the invention usually comprises from 10 to 70% by weight, preferably 20 to 60% by weight, more preferably 30 to 50% by weight, in particular 35 to 45% by weight, of atazanavir. The quantity indicated here relates to the weight of atazanavir base. Thus, in the case of the preferably used bisulphate, the weight of H₂SO₄ must be subtracted.

The adhesion enhancer is generally a substance which is suitable for fixing atazanavir in a compacted or compressed form. Addition of the adhesion enhancer usually leads to an increase in the interparticle surfaces at which bonds can form (e.g. during the compression procedure). Furthermore, adhesion enhancers are characterized by increasing the plasticity of the tableting mixture, resulting in solid tablets being produced by the compression.

In a possible embodiment, the adhesion enhancer is a polymer. The term “adhesion enhancer” further comprises also substances that behave similarly to polymers. The adhesion enhancer furthermore comprises solid, non-polymeric compounds which preferably have polar side groups. Examples of these are sugar alcohols or disaccharides.

The adhesion enhancer used in the context of the present invention is preferably a polymer having a glass transition temperature (Tg) of higher than 15° C., more preferably from 40° C. to 150° C., in particular from 50° C. to 100° C.

The “glass transition temperature” (Tg) denotes the temperature at which amorphous or partly crystalline polymers change from the solid state into the liquid state. This is accompanied by a distinct change in physical parameters, for example hardness and elasticity. A polymer is usually glass-like and hard below the Tg and changes into a rubber-like to viscous state above the Tg. The glass transition temperature is determined in the context of the present invention by means of differential scanning calorimetry (DSC). A Mettler Toledo DSC 1 instrument may be employed for this, for example. A heating rate of 1-20° C./min, preferably 5-15° C./min and/or a cooling rate of 5-25° C./min, preferably 10-20° C./min is employed.

The polymer usable as adhesion enhancer has also a weight-average molecular weight of preferably from 1000 to 500 000 g/mol, more preferably from 2000 to 90 000 g/mol. The weight-average molecular weight is usually determined by means of gel permeation chromatography. If the polymer used for preparing the intermediate is dissolved in water at 2% by weight, the resulting solution exhibits a viscosity of preferably from 0.1 to 20 mPa·s, more preferably from 0.5 to 12 mPa·s, in particular from 1.0 to 8.0 mPa·s, measured at 25° C., and determined preferably according to Ph. Eur., 6th edition, chapter 2.2.10.

Preference is given to using hydrophilic polymers for preparing the intermediate, meaning polymers having hydrophilic groups. Examples of suitable hydrophilic groups are hydroxyl, alkoxy, acrylate, methacrylate, sulphonate, carboxylate and quaternary ammonium groups, with hydroxy groups being preferred.

The intermediate according to the invention may comprise, for example, the following polymers as adhesion enhancers: polysaccharides such as hydroxypropyl-methylcellulose (HPMC), carboxymethylcellulose (CMC, in particular sodium and calcium salts), ethylcellulose, methylcellulose, hydroxyethylcellulose, ethylhydroxyethylcellulose, hydroxypropylcellulose (HPC); microcrystalline cellulose, with preference being given to using types with a higher maximum moisture content of up to 7%, silicon-modified microcrystalline cellulose (e.g. Prosolv®), guar flour, alginic acid and/or alginates; synthetic polymers such as polyvinylpyrrolidone (Povidone), polyvinyl acetate (PVAC), polyvinyl alcohol (PVA), polymers of acrylic acid and salts thereof, polyacrylamide, polymethacrylates, vinylpyrrolidone-vinyl acetate copolymers (for example Kollidon® VA64, BASF), polyalkylene glycols such as polypropylene glycol or preferably polyethylene glycol, co-block polymers of polyethylene glycol, in particular co-block polymers of polyethylene glycol and polypropylene glycol (Pluronic®, BASF) and mixtures of said polymers. It is furthermore possible to use starch, starch derivatives, treated starch and pre-gelatinized starch as adhesion enhancers. It is likewise possible to use crosslinked polyvinylpyrrolidone as adhesion enhancer, in particular in micronized form (D₅₀ 0.1-10 μm), e.g. sold as Kollidon® CL-M.

The abovementioned polyvinylpyrrolidone has a weight-average molecular weight of preferably from 10 000 to 60 000 g/mol, in particular 12 000 to 40 000 g/mol. It is also possible to use copolymers of vinylpyrrolidone and vinyl acetate, in particular those with a weight-average molecular weight of from 40 000 to 70 000 g/mol, and/or polyethylene glycol, in particular with a weight-average molecular weight of from 2000 to 10 000 g/mol, and also HPMC, in particular with a weight-average molecular weight of from 20 000 to 90 000 g/mol, and/or preferably a proportion of methyl groups of from 10 to 35% and a proportion of hydroxy groups of from 1 to 35%. It is also possible to preferably use microcrystalline cellulose, in particular one with a specific surface of from 0.7 to 5.0, more preferably from 1.5 to 3.0, m²/g. The specific surface was determined by means of the gas adsorption method according to Brunauer, Emmet and according to Ph. Eur., 6th edition, 2.9.26., Method 1. Finally, preference is given to using pre-gelatinized starch.

The adhesion enhancer further comprises also solid, non-polymeric compounds which preferably have polar side groups. Examples of these are sugar alcohols or disaccharides. Examples of particularly suitable sugar alcohols and/or disaccharides are lactose, mannitol, sorbitol, xylitol, isomalt (disaccharide alcohol in a 3:1 ratio of 6-O-alpha-D-glucopyranosyl-D-sorbitol and 1-O-alpha-D-glucopyranosyl-D-mannitol dihydrate), glucose, fructose, maltose, and mixtures thereof. The term sugar alcohols here also comprises monosaccharides. In the case of lactose, preference is given to using the alpha-lactose monohydrate, in particular crystalline alpha-lactose monohydrate with a tapped density of from 450 to 550 g/l and a bulk density of from 550 to 680 g/l. The average particle size (D₅₀) is preferably between 60 and 200 μm, particularly preferably between 100 and 200 μm. It is likewise also possible to use modified lactose, in particular compositions of alpha-lactose monohydrate and corn starch.

Preference is in particular given to using as adhesion enhancer lactose, compositions of alpha-lactose monohydrate and corn starch and/or isomalt.

Mixtures of said adhesion enhancers are also possible.

The oral dosage form according to the invention usually contains adhesion enhancer in an amount of from 10 to 80% by weight, preferably from 15 to 70% by weight, more preferably from 20 to 60% by weight, particularly preferably from 25 to 50% by weight, based on the total weight of the dosage form.

The present invention makes use of atazanavir and adhesion enhancer in an amount, wherein the atazanavir to adhesion enhancer weight ratio is usually 1:10 to 10:1, preferably 5:1 to 1:7, more preferably 3:1 to 1:5, still more preferably 2:1 to 1:3, in particular 1:1 to 1:2.

The process according to the invention can generally be carried out by way of two embodiments, namely by means of dry compaction and by means of direct compression. Preference is given to dry compaction in the process according to the invention. Both embodiments are carried out in the absence of solvent.

One aspect of the present invention therefore relates to a dry compaction process comprising the steps
(a) mixing of atazanavir with an adhesion enhancer and optionally further pharmaceutical excipients;
(b) compaction to give a slug;
(c) granulation of the slug;
(d) compression of the resulting granules to give tablets, where appropriate with addition of further pharmaceutical excipients; and
(e) optionally covering of the tablets with a film.

Step (a) comprises mixing atazanavir and adhesion enhancer, and optionally further pharmaceutical excipients (described below). The mixing may be carried out in conventional mixers. For example, mixing can be carried out in mechanical mixers or gravity mixers, for example by means of Turbula® T10B (Bachofen AG, Switzerland). Alternatively, it is possible for atazanavir to be mixed initially only with part of the excipients (e.g. 50 to 95%) prior to compaction (b), and for the remaining part of the excipients to be added after the granulation step (c). In the case of multiple compaction, the excipients should be admixed preferably prior to the first compaction step, in between multiple compaction steps or after the last granulation step.

The mixing conditions in step (a) and/or the compacting conditions in step (b) are usually chosen such that at least 30% of the surface of the resulting atazanavir particles are covered with adhesion enhancer, more preferably that at least 50% of the surface, particularly preferably at least 70% of the surface, in particular at least 90% of the surface, are covered.

Step (b) of the process according to the invention comprises compacting the mixture of step (a) to give a slug. This is a dry compaction, i.e. said compaction is preferably carried out in the absence of solvents, more specifically in the absence of organic solvents.

Compaction is preferably carried out in a roll granulator.

The rolling force is usually 5 to 70 kN/cm, preferably 10 to 60 kN/cm, more preferably 15 to 50 kN/cm.

The gap width of the roll granulator is, for example, 0.8 to 5 mm, preferably 1 to 4 mm, more preferably 1.5 to 3 mm, in particular 1.8 to 2.8 mm.

The compaction device used preferably has a cooling device. In particular, cooling is carried out in such a way that the temperature of the compacted material does not exceed 50° C., in particular 40° C.

Step (c) of the process comprises granulating the slug. Said granulation may be carried out using processes known in the prior art. For example, granulation is carried out using a Comil® U5 (Quadro Engineering, USA) apparatus, preferably with subsequent sieving.

In an alternative embodiment, compaction may be carried out in a compactor, with the slug being granulated through an integrated sieve. Thus a preferred embodiment comprises carrying out steps (b) and (c) in a single apparatus.

In a preferred embodiment, the granulation conditions are chosen such that the resulting intermediates (granules) have a particle size distribution D₅₀ value of from 50 to 800 μm, more preferably from 90 to 630 μm, still more preferably 150 to 450 μm, in particular from 180 to 350 μm.

Furthermore, the granulation conditions are preferably chosen such that the resulting granules have a bulk density of from 0.2 to 0.85 g/ml, more preferably 0.3 to 0.8 g/ml, in particular 0.4 to 0.7 g/ml. The Hausner factor is usually in the range from 1.02 to 1.3, more preferably from 1.04 to 1.20, and in particular from 1.04 to 1.15. “Hausner factor” here means the ratio of tapped density to bulk density. Bulk and tapped densities are determined according to Ph. Eur 4.0, 2.9.15.

In a preferred embodiment, granulation is carried out in a sieving mill. In this case, the mesh width of the sieve insert is usually 0.1 to 5 mm, preferably 0.5 to 3 mm, more preferably 0.75 to 2 mm, in particular 0.8 to 1.8 mm.

In a preferred embodiment, the process is adapted such that multiple compaction takes place, with the granules resulting from step (c) being returned one or more times to the compaction (b). The granules from step (c) are returned preferably 1 to 5 times, in particular 2 to 3 times.

The granules resulting from step (c) can be processed to give pharmaceutical dosage forms. To this end, the granules are filled into sachets or capsules, for example. The invention therefore also relates to capsules and sachets comprising a granulated pharmaceutical composition which is obtainable by the dry granulation process according to the invention.

The granules resulting from step (c) are preferably compressed to give tablets (=step (d) of the process according to the invention).

Step (d) comprises a compression to give tablets. Said compression may be carried out using tableting machines known in the prior art. The compression is preferably carried out in the absence of solvents.

Examples of suitable tableting machines are excenter presses or rotary presses. An example of a rotary press that may be used is a Fette® 102i (Fette GmbH, Germany). In the case of rotary presses, a pressing force of from 2 to 40 kN, preferably from 2.5 to 35 kN, is usually applied. In the case of excenter presses (e.g. Korsch® EKO) a pressing force of from 1 to 20 kN, preferably from 2.5 to 10 kN, is usually applied.

In step (d) of the process pharmaceutical excipients may optionally be added to the granules of step (c). The amounts of excipients added in step (d) are usually a function of the type of tablet to be produced and of the amount of excipients previously added in steps (a) and (b).

The optional step (e) of the process according to the invention comprises covering the tablets of step (d) with a film. This may involve applying the processes common in the prior art for covering tablets with a film.

Preference is given to using macromolecular substances for applying a film, for example modified celluloses, polymethacrylates, polyvinylpyrrolidone, polyvinyl acetate phthalate, zein, and/or shellack or natural gums such as carrageenan, for example.

Preference is given to using HPMC, in particular HPMC with a weight average molecular weight of from 10 000 to 150 000 g/mol and/or an average degree of substitution on —OCH₃ groups of from 1.2 to 2.0.

The coating layer thickness is preferably 2 to 100 μm, in particular 5 to 50 μm.

A further aspect of the present invention, besides the above-described dry compaction and granulation processes, is a compacted intermediate containing atazanavir. The invention therefore further relates to an intermediate obtainable by dry compaction of atazanavir together with an adhesion enhancer.

Regarding the properties of the atazanavir to be used and of the adhesion enhancer to be used, reference is made to the discussions above. The intermediate according to the invention may be produced by steps (a) and (b) of the process according to the invention discussed above.

The compaction conditions for producing the intermediate according to the invention are usually chosen such that the intermediate according to the invention forms a compacted material (slug), with the apparent density of the compacted material being 0.8 to 1.3 g/cm³, preferably 0.9 to 1.20 g/cm³, in particular 1.01 to 1.15 g/cm³.

The apparent density of the compacted material (and thus of the intermediate) is calculated as follows:

apparent density_{slug}=mass_{slug}/volume_{slug}

In the context of the present invention, the apparent density is determined by using the throughput method, in particular with the use of a roll compactor or roll granulator.

Establishing the rate of throughput in dry compaction:

The measurement is carried out according to the weighing principle by collecting the compacted mass (=slug from step (b)) under otherwise constant conditions within a defined period of time and precisely measuring said mass by weighing. A possible increase in moisture of the compacted material is then corrected mathematically.

For this purpose, once the compactor operates at a constant roller speed, gap width and compacting force, i.e. the starting phase has been succeeded by the production phase, the compacted material is collected completely and without loss in a pharmaceutically suitable container, and the corresponding process time is recorded. To this end, a stopwatch is used to establish a period of 2 minutes, corresponding to 120 s, and the compacted material collected within this period is used for the measurement. The compacted material is then measured by weighing and the moisture is determined (halogen lamp moisture analyser). The weighed mass is corrected by the moisture difference before and after compaction, and the mass flow is then calculated by dividing the mass in kg by the time in minutes.

Result: Throughput of compacted material in kg/min. The throughput of compacted material in kg/h is obtained by multiplying by 60.

Measuring principle of a halogen lamp moisture analyser:

The method of measurement is thermogravimetry, i.e. a defined mass is thermally stimulated and possibly releases water. The change in weight is a measure of the moisture present (see e.g. Mettler-Toledo: Halogen Moisture Analyzer HG 63).

The apparent density is then calculated by the following formula:

apparent density=mass throughput/volume throughput; where

volume throughput=roller speed×roller width×roller diameter×number π(pi)×gap width.

The slug resulting in process step (b) can furthermore be characterized by porosity. It usually has a porosity of between 0.16 and 0.45, preferably between 0.25 and 0.43, particularly preferably between 0.28 and 0.40.

The typical throughput through the compactor is usually 12-45 kg/h, preferably 15-30 kg/h. To this end, preference is given to using the abovementioned gap width (in particular 2.5 to 4.5 mm) and a roller width of 100 mm.

The porosity is calculated according to the formula:

Porosity epsilon=1−(true density of starting material/apparent density of slug)

The starting material is the mixture obtained in process step (a). The true density may be determined using a gas pycnometer. Said gas pycnometer is preferably a helium pycnometer, more specifically use is made of the instrument AccuPyc 1340 Helium Pyknometer manufactured by Micromeritics, Germany.

Example of Calculating Porosity:

| | | | | | | | | |
|---|---|---|---|---|---|---|---|---|
| True density of | 1.4 | g/cm³ | 1.6 | g/cm³ | 1.4 | g/cm³ | 1.4 | g/cm³ |
| starting | | | | | | | | |
| material | | | | | | | | |
| Throughput | 15 | kg/h | 15 | kg/h | 45 | kg/h | 15 | kg/h |
| Gap width | 3.24 | mm | 3.24 | mm | 3.24 | mm | 5.00 | mm |
| Roller speed | 1 | /min | 1 | /min | 3 | /min | 1 | /min |
| Roller diameter | 250 | mm | 250 | mm | 250 | mm | 250 | mm |
| Roller width | 100 | mm | 100 | mm | 100 | mm | 100 | mm |

Results:

| Apparent | 0.982 g/cm³ | 0.982 g/cm³ | 0.982 g/cm³ | 0.637 g/cm³ |
|---|---|---|---|---|
| density | | | | |
| Porosity | 0.298 | 0.386 | 0.298 | 0.545 |

Preference is given to choosing type and amount of the adhesion enhancer (and, where appropriate, of the other pharmaceutical excipients) in such a way that the resulting intermediate (and also the resulting oral dosage form) are storage-stable. “Storage-stable” means that the proportion of crystalline atazanavir—based on the total amount of atazanavir—is at least 60% by weight, preferably at least 75% by weight, more preferably at least 85% by weight, in particular at least 95% by weight, in the intermediate according to the invention after 3 years of storage at 25° C. and 60% relative humidity.

An exemplary formulation for the oral dosage form according to the invention may comprise:
atazanavir in an amount of from 10 to 70% by weight, preferably 20 to 60% by weight, particularly preferably 30 to 50% by weight, in particular preferably 35 to 45% by weight,
adhesion enhancer in an amount of from 10 to 80% by weight, preferably 15 to 70% by weight, particularly preferably 20 to 60% by weight, in particular preferably 25 to 50% by weight,
optionally disintegrant in an amount of from 0 to 25% by weight, preferably 2 to 15% by weight, particularly preferably 5 to 12% by weight, and
optionally lubricant in an amount of from 0 to 5% by weight, preferably 0.1 to 4% by weight,
optionally flow agent in an amount of from 0 to 5% by weight, preferably 0.5 to 3% by weight, in each case based on the total weight of the formulation.

The intermediates according to the invention may (as described above under step (c) of the process according to the invention) be comminuted, for example granulated. The intermediate according to the invention is usually used for preparing a pharmaceutical formulation. To this end, one embodiment comprises filling the intermediate—where appropriate together with further excipients (see discussions below)—into single- and multiple-dose containers, preferably sachets and stick packs. Consequently, the invention also relates to single- and multiple-dose containers, preferably sachets and stick packs, containing the granules according to the invention.

However, preference is given in another embodiment to the intermediate according to the invention being compressed to give tablets—as described above in step (d) of the process according to the invention.

In the case of direct compression, only steps (a) and (d) and optionally (e) of the above-described process are carried out. The invention therefore relates to a process comprising the steps
(a) mixing of atazanavir with an adhesion enhancer and optionally further pharmaceutical excipients; and
(d) direct compression of the resulting mixture to give tablets, and
(e) optionally covering of the tablets with a film.

The discussions above regarding steps (a), (d) and (e) in principle also apply to direct compression.

In a preferred embodiment, step (a) in the case of direct compression comprises milling atazanavir and adhesion enhancer together. Further pharmaceutical excipients may optionally be added.

The milling conditions are usually chosen such that at least 30% of the surface of the resulting atazanavir particles, more preferably at least 50% of the surface, particularly preferably at least 70% of the surface, in particular at least 90% of the surface, are covered with adhesion enhancer.

Milling is generally carried out in common milling devices, for example in a ball mill, air jet mill, pin mill, classifier mill, cross-arm beater mill, disc mill, mortar mill, rotor mill. The milling time is usually 0.5 minutes to 1 hour, preferably 2 minutes to 50 minutes, more preferably 5 minutes to 30 minutes.

In the case of direct compression, preference is given to employing in step (d) a mixture, wherein the particle sizes of active compound and excipients match one another. Preference is given to a mixture comprising atazanavir, adhesion enhancer and optionally further pharmaceutical excipients in particulate form with a D₅₀ value of from 50 to 250 μm, more preferably from 60 to 180 μm, in particular from 70 to 130 μm. The particle size distribution of the mixture may be monomodal or bimodal. In a preferred embodiment the particle size distribution of the mixture is monomodal. “Monomodal” here means that the particle size distribution, when depicted in a histogram and/or a frequency distribution curve, has only one maximum. Correspondingly, “bimodal” here means that the particle size distribution, when depicted in a histogram and/or a frequency distribution curve, has two maxima.

Both in the case of dry compaction and in the case of direct compression it is possible to use, in addition to atazanavir and adhesion enhancer, still further pharmaceutical excipients. These are the excipients known to the skilled worker, in particular those described in the European Pharmacopoeia. The same applies to the use of the intermediate according to the invention for filling single- and multiple-dose containers.

Examples of excipients used are disintegrants, anticaking agents, emulsifiers, pseudoemulsifiers, fillers, additives for improving powder flowability, glidants, wetting agents, gel formers, lubricants and/or stabilizing agents. It is possible, where appropriate, to use still further excipients.

Substances generally referred to as disintegrants are those which accelerate disintegration of a dosage form, more specifically a tablet, after it has been introduced into water. Examples of suitable disintegrants are organic disintegrants such as carrageenan, croscarmellose and crospovidone. Use is likewise made of alkaline disintegrants. Alkaline disintegrants mean disintegrants which generate a pH above 7.0 when dissolved in water.

Preferably, inorganic alkaline disintegrants may be used, in particular salts of alkali metals and alkaline earth metals. Preferred mention may be made here of sodium, potassium, magnesium and calcium. Preferred anions are carbonate, hydrogen carbonate, phosphate, hydrogen phosphate and dihydrogen phosphate. Examples are sodium hydrogen carbonate, sodium hydrogen phosphate, calcium hydrogen carbonate and the like.

Disintegrants are used in the present case usually in an amount of from 0 to 25% by weight, more preferably from 1 to 15% by weight, particularly preferably 3 to 12% by weight, based on the total weight of the oral dosage form.

The oral dosage form according to the invention may likewise contain flow agents. One example of an additive for improving powder flowability is disperse silicon dioxide, known under the trade name Aerosil®, for example. Preference is given to using silicon dioxide having a specific surface of from 50 to 400 m²/g, more preferably from 100 to 250 m²/g, determined by gas adsorption according to Ph. Eur., 6th edition, 2.9.26, Method 1.

Additives for improving powder flowability are usually used in an amount of from 0.1 to 3% by weight, based on the total weight of the oral dosage form.

Lubricants may also be used. Lubricants generally serve to reduce sliding friction. More specifically, it is intended to reduce the sliding friction which exists during tableting firstly between the pouches moving up and down in the die bore and the die wall and secondly between tablet band and die wall. Examples of suitable lubricants are stearic acid, adipic acid, sodium stearyl fumarate and/or magnesium stearate.

Lubricants are usually used in an amount of from 0.1 to 5% by weight, preferably from 0.5 to 4% by weight, based on the total weight of the oral dosage form.

It is also possible, in a further embodiment, to use stabilizing agents as pharmaceutical excipient. The term “stabilizing agent” here comprises means which serve to prevent a conversion or partial conversion of the polymorphic forms of atazanavir into one another, to prevent a conversion of the crystalline state into the amorphous state during processing and/or storage.

Preference is given here to using as stabilizing agents inorganic acids or carboxylic acids such as, for example, mono-, di- or tricarboxylic acids and/or their salts, for example with a pKa from 1 to 5. Particular preference is given to using here tricarboxylic acids, for example with a pKa₁ from 2 to 4, in particular citric acid.

Stabilizing agents are usually used in an amount of from 0.1 to 15% by weight, preferably from 1 to 12% by weight, particularly preferably from 5 to 10% by weight, based on the total weight of the oral dosage form.

It is in the nature of pharmaceutical excipients sometimes to have multiple functions in a pharmaceutical formulation. For unambiguous delimitation in the context of the present invention, therefore the fiction preferably applies that a substance used as a particular excipient is not also employed as further pharmaceutical excipient at the same time. Thus, for example, microcrystalline cellulose, if used as adhesion enhancer, is not also used as disintegrant, although microcrystalline cellulose exhibits a certain disintegrant action.

The ratio of active compound to excipients is preferably chosen in such a way that the formulations resulting from the process according to the invention (i.e., for example, the tablets according to the invention) contain 20 to 60% by weight, more preferably 30 to 50% by weight, in particular 35 to 45% by weight, atazanavir and 40 to 80% by weight, more preferably 50 to 70% by weight, in particular 55 to 65% by weight, pharmaceutically compatible excipients.

This information regards the amount of adhesion enhancer used in the process according to the invention and/or for preparing the intermediate according to the invention to be an excipient. That is to say the amount of active compound relates to the amount of atazanavir present in the formulation.

The formulations according to the invention (i.e. the tablets according to the invention or the granules according to the invention resulting from step (c) of the process according to the invention, with which granules stick packs or sachets can be filled, for example) have been shown to be able to serve both as dosage form with immediate release (abbreviated “IR”) and as dosage form with modified release (abbreviated “MR”).

In a preferred embodiment, the oral dosage form according to the invention is a dosage form with immediate release (abbreviated “IR”), in particular in the form of an oral tablet.

The release profile of the oral dosage form according to the invention has usually a released active compound content of at least 30%, preferably at least 60%, in particular at least 90%, after 10 minutes, according to the FDA method. The active compound release here is determined by means of the FDA method at 50 rpm, in 1000 ml of 0.025 N HCl at 37° C., using a paddle apparatus.

The above-mentioned pharmaceutical excipients can be employed in the two preferred embodiments (dry compaction and direct compression). Preference is further given to the tableting conditions being chosen in both embodiments of the process according to the invention in such a way that the resulting tablets have a tablet height to weight ratio of from 0.005 to 0.3 mm/mg, particularly preferably 0.05 to 0.2 mm/mg.

The process according to the invention is preferably carried out such that the tablet according to the invention contains atazanavir in an amount of more than 20 mg to 500 mg, more preferably from 50 mg to 400 mg, in particular 50 mg to 300 mg. The invention thus relates to tablets containing 100 mg, 150 mg, 200 mg, 300 mg, 400 mg, or 500 mg of atazanavir.

The resulting tablets further have a hardness of preferably from 20 to 200 N, particularly preferably from 30 to 150 N, in particular 50 to 85 N. The hardness is determined according to Ph. Eur. 6.0, section 2.9.8.

Moreover, the resulting tablets exhibit a friability preferably of less than 3%, particularly preferably of less than 2%, in particular less than 1%. Friability is determined according to Ph. Eur. 6.0, section 2.9.7.

Finally, the tablets according to the invention usually have a content uniformity of from 95 to 105%, preferably from 98 to 102%, in particular from 99 to 101%, of the average content. (That is to say all tablets have an active compound content of between 95 and 105%, preferably between 98 and 102%, in particular between 99 and 101%, of the average active compound content.) Content uniformity is determined according to Ph. Eur. 6.0, section 2.9.6.

The information above regarding hardness, friability, content uniformity and release profile here relates preferably to the tablet for an IR formulation, which tablet is not film-covered. The weight information, unless stated otherwise, likewise relates to the tablet which is not film-covered. In the case of capsules, sachets or stick packs, the weight information relates to the formulation introduced therein, i.e. without the weight of the capsule, the sachet envelope or the stick pack envelope.

The tablets produced by the process according to the invention may be tablets which are swallowed in unchewed form (without a film or preferably covered with a film). They may likewise be dispersible tablets. “Dispersible tablet” here means a tablet for producing an aqueous suspension for administration.

In the case of tablets which are swallowed in unchewed form, preference is given, as discussed above under step (e), to said tablets being covered with a film layer. The sachet formulation may moreover include an effervescent material which consists of a mixture of acid and CO₂ former such as, for example, sodium hydrogen carbonate, for example in a 1:2 ratio, and preferably constitutes 5 to 15% by weight, for example about 10% by weight, of the total amount.

As discussed above, the invention relates to not only the process according to the invention but also the tablets produced by said process. The tablets produced by the dry compaction process according to the invention were also shown to have preferably a bimodal pore size distribution. The invention thus relates to tablets comprising atazanavir or a pharmaceutically compatible salt thereof and adhesion enhancer and optionally pharmaceutically compatible excipients, which tablets have a bimodal pore size distribution.

Tablets with bimodal pore size distribution have been shown to exhibit an advantageous release profile and rise-in-level behaviour.

Said tablet according to the invention is provided when the granules of process step (c) are compressed. This compressed material consists of solid and pores. The pore structure may be characterized in more detail by determining the pore size distribution.

The pore size distribution was determined by means of mercury porosimetry. Mercury porosimetry measurements were carried out using the porosimeter “Poresizer” from Micromeritics, Norcross, USA. The pore sizes were calculated here on assuming a mercury surface tension of 485 mN/m. From the cumulative pore volume, the pore size distribution was calculated as summed distribution or proportion of pore fractions in percent. The average pore diameter (4V/A) was determined from the total specific mercury intrusion volume (Vtotᵢₙₜ) and the total pore area (Atotₚₒᵣ), according to the following equation.

$$4 V \text{/} A = \frac{4 \cdot {Vtot}_{int} [ ml \text{/} g ]}{{Atot}_{por} [ {m}^{2} \text{/} g ]} .$$

“Bimodal pore size distribution” means that the pore size distribution has two maxima. Said two maxima are not necessarily separated by a minimum but a head-shoulder formation is also considered bimodal for the purpose of the invention.

The examples below are intended to illustrate the invention. All examples preferably employ atazanavir by way of atazanavir bisulphate, with the indicated amount referring to the amount of atazanavir in the form of the free base.

### EXAMPLES

a) Production by Direct Compression

### Example 1

| | | |
|---|---|---|
| Atazanavir bisulphate | 150 mg | (calculated for the free base) |
| Modified lactose* | 170 mg | |
| Crosslinked PVP | 20 mg | |
| Silicon dioxide | 6 mg | |
| Citric acid | 30 mg | |
| Magnesium stearate | 5 mg | |

*Modified lactose: spray-dried compound consisting of 85% alpha-lactose monohydrate (Ph. Eur./USP-NF) and 15% corn starch (Ph. Eur./USP-NF) (StarLac ®).

Atazanavir was mixed with modified lactose, crosslinked PVP, silicon dioxide and citric acid and applied to the sieve 630 μm. This was followed by pre-mixing the mixture in a gravity mixer (Turbula® T10B) for 15 minutes. Magnesium stearate was added to the mixture, all of which was then mixed again in the gravity mixer for another 3 min. The finished mixture was compressed on an eccentric press (Korsch® EKO) with 10 mm round biconvex punches. The tablets had a hardness of approx. 50-85 N.

### Example 2

| | | |
|---|---|---|
| Atazanavir bisulphate | 150 mg | (calculated for the free base) |
| Isomalt | 170 mg | |
| Crosslinked PVP | 20 mg | |
| Silicon dioxide | 6 mg | |
| Citric acid** | 30 mg | (coated with 2 mg of hypromellose |
| | | (Methocel ® E5)) |
| Magnesium stearate | 5 mg | |

**Citric acid is coated beforehand with hypromellose (Methocel ® E5) in the WSG (Glatt GPCG 3.1). Hypromellose (Methocel ® E5) is dissolved in 96% (v/v) strength ethanol solution and then sprayed in the WSG on to the citric acid already present. Mixture: coating solution: 1000.0 g of ethanol 96%(v/v), 500.0 g of hypromellose (Methocel ® E5).

Atazanavir was mixed with isomalt, crosslinked PVP, silicon dioxide and citric acid and applied to the sieve 630 μm. This was followed by pre-mixing the mixture in a gravity mixer (Turbula® T10B) for 15 minutes. Magnesium stearate was added to the mixture, all of which was then mixed again in the gravity mixer for another 3 min. The finished mixture was compressed on an eccentric press (Korsch® EKO) with 10 mm round biconvex punches. The tablets had a hardness of approx. 50-85 N.

b) Production by Dry Compaction

### Example 3

| | | |
|---|---|---|
| Atazanavir bisulphate | 150 mg | (calculated for the free base) |
| Microcrystalline cellulose | 70 mg | |
| Lactose monohydrate | 100 mg | |
| Cross-linked PVP | 20 mg | |
| Silicon dioxide | 6 mg | |
| Citric acid | 30 mg | |
| Magnesium stearate | 5 mg | |

Atazanavir was compacted with in each case ⅔ of the total amounts of microcrystalline cellulose and lactose monohydrate, half of the crosslinked PVP and the total amount of citric acid and applied via the sieve 630 μm. This was followed by pre-mixing the mixture in a gravity mixer (Turbula® T10B) for 15 minutes. Magnesium stearate was added to the mixture, all of which was then mixed again in the gravity mixer for another 3 min. The finished mixture was compressed on an eccentric press (Korsch® EKO) with 10 mm round biconvex punches. The tablets had a hardness of approx. 50-85 N.

### Example 4

| | | |
|---|---|---|
| Atazanavir bisulphate | 150 mg | (calculated for the free base) |
| Microcrystalline cellulose | 70 mg | |
| Lactose monohydrate | 100 mg | |
| Cross-linked PVP | 20 mg | |
| Silicon dioxide | 6 mg | |
| Citric acid** | 30 mg | (coated with 2 mg of |
| | | Methocel ® E5) |
| Magnesium stearate | 5 mg | |

**Citric acid was coated beforehand with hypromellose (Methocel ® E5) in the WSG (Glatt GPCG 3.1). Hypromellose (Methocel ® E5) was dissolved in 96% (v/v) strength ethanol solution and then sprayed in the WSG on to the citric acid already present.

Atazanavir was compacted with in each case ⅔ of the total amounts of microcrystalline cellulose and lactose monohydrate, half of the crosslinked PVP and the total amount of citric acid** and applied via the sieve 630 μm. This was followed by pre-mixing the mixture in a gravity mixer (Turbula® T10B) for 15 minutes. Magnesium stearate was added to the mixture, all of which was then mixed again in the gravity mixer for another 3 min. The finished mixture was compressed on an eccentric press (Korsch® EKO) with 10 mm round biconvex punches. The tablets had a hardness of approx. 50-85 N.

1. Process for producing oral dosage forms, comprising atazanavir and adhesion enhancers, wherein said dosage forms are produced by means of dry compaction or by means of direct compression, and the atazanavir to adhesion enhancer weight ratio is from 5:1 to 1:7.

2. Process according to claim 1, comprising the steps
(a) mixing of atazanavir with an adhesion enhancer and optionally further pharmaceutical excipients;
(b) compaction to give a slug;
(c) granulation of the slug;
(d) compression of the resulting granules to give tablets, where appropriate with addition of further pharmaceutical excipients; and
(e) optionally covering of the tablets with a film, with the granulation conditions in step (c) being chosen such that the D₅₀ value of the particle size distribution of the granules lies between 100 and 450 μm.

3. Process according to claim 2, wherein the compaction (b) is carried out in a roll granulator and the rolling force is from 2 to 70 kN/cm.

4. Process according to claim 1, comprising the steps
(a) mixing of atazanavir with an adhesion enhancer and optionally further pharmaceutical excipients; and
(d) direct compression of the resulting mixture to give tablets, and
(e) optionally covering of the tablets with a film.

5. Process according to claim 4, wherein the mixture resulting from step (a) has a particle size distribution D₅₀ value of from 50 to 250 μm.

6. Process according to claim 1, wherein a stabilizing agent is added.

7. Process according to claim 6, where the stabilizing agent is citric acid.

8. Process according to claim 1, wherein atazanavir is used in an amount of from 20 to 60% by weight, based on the total weight of all substances used.

9. Process according to claim 1, wherein particulate atazanavir with a particle size distribution D₅₀ value of from 5 to 150 μm is used.

10. Tablets obtainable according to claim 1.

11. Tablets according to claim 10 having a friability of less than 3%, a content uniformity of from 95 to 105% and a hardness of from 30 to 200 N, said tablets comprising from 50 to 300 mg of atazanavir.

12. Tablet comprising atazanavir and adhesion enhancers, with the atazanavir to adhesion enhancer weight ratio being from 5:1 to 1:7 and said tablet having a bimodal pore size distribution.

13. Intermediate obtainable by dry compaction of atazanavir together with an adhesion enhancer, with the atazanavir to adhesion enhancer weight ratio being from 5:1 to 1:7.

14. Intermediate according to claim 13, wherein the density of the intermediate is from 0.8 to 1.3 g/cm³.

15. Sachet or stick pack comprising an intermediate according to claim 13.
